(19) United States
(12) Patent Application Publication
(10) Pub. No.: US 2015/0203420 A1
(43) Pub. Date: Jul. 23, 2015

(54) REMOVAL OF METALS FROM LIQUID PYROLYSIS OIL

(71) Applicants: Guangci Zhou, El Cerrito, CA (US); Stephen H. Roby, Hercules, CA (US); Christopher W. Kuehler, Larkspur, CA (US)

(72) Inventors: Guangci Zhou, El Cerrito, CA (US); Stephen H. Roby, Hercules, CA (US); Christopher W. Kuehler, Larkspur, CA (US)

(73) Assignee: Chevron U.S.A. Inc., San Ramon, CA (US)

(21) Appl. No.: 14/158,089

(22) Filed: Jan. 17, 2014

(51) Int. Cl. C07C 7/12

(52) U.S. Cl. CPC C07C 7/12

(57) ABSTRACT

The present disclosure generally relates to removing metals from liquid-phase pyrolysis oil, such as at or near room temperatures. Specifically, some embodiments of the disclosure relate to a method and a system for removing metals from pyrolysis oil using acidic ion-exchange resins. One embodiment relates to a method for removing metals from pyrolysis oil comprising combining pyrolysis oil and an organic solvent to form a pyrolysis oil mixture and removing metal from the pyrolysis oil mixture to produce a reduced metal content pyrolysis oil mixture. The metal can then be removed by using a strongly acidic ion-exchange resin.

### TECHNICAL FIELD

The present disclosure generally relates to removing metals from liquid pyrolysis oil. Specifically, some embodiments of the disclosure relate to a method and a system for removing metals from pyrolysis oil using acidic ion-exchange resins at lower temperatures, such as less than 100° C.

### BACKGROUND

Pyrolysis oil is made from biomass, waste plastics, and other such carbon based material by the thermal decomposition of the material in the absence of oxygen. In the example of biomass, the pyrolysis splits the cellulose and lignin chains into smaller units. Pyrolysis oil mainly comprises water (20-28%), suspended solids and pyrolytic lignin (22-36%), hydroxyacetaldehyde (8-12%), and a host of other components such as levoglucosan, acetic acid, acetol, cellubiosan, glyoxal, formaldehyde, and formic acid. The oxygen content of the pyrolysis oil is approximately 40%. The pH of pyrolysis oil is between 1.5 and 3.8, which can require special processing equipment. Fast pyrolysis oil is the condensed product of pyrolysis gases and organic vapors from materials that are rapidly heated at temperatures around 500 degrees Celsius without oxygen.

Pyrolysis oil can either be directly burned as fuel or used as a potential feedstock in petroleum refineries. It is estimated that the pyrolysis oil might replace up to 60% of transportation fuels. It would significantly reduce the dependency on petroleum crude oil. Oil refiners would like to use pyrolysis oils as crude oil substitutes or extenders, blending the pyrolysis oil into conventional crude oil, and then processing the mixture in existing plants. However, the low pH, enormous oxygen content of non-water components, and large metal concentrations make co-processing the pyrolysis oil difficult. Refining equipment is subject to corrosion by low molecular weight organic acids. Additionally, pyrolysis oil has as high as 1700 ppm contaminant metals, such as calcium, potassium, magnesium, and sodium. These metals poison the catalysts used in refining processes and, therefore, must be removed in advance. The metals in pyrolysis oil could be removed by adsorbents or ion-exchange resins. However, the nature of pyrolysis oil makes the metal removal process difficult. For example, pyrolysis oil is highly viscous. Its viscosity at 20 degrees Celsius can range from 400 to 5000 cSt. Although heating can reduce pyrolysis oil viscosity, the poor thermal stability of pyrolysis oil limits the temperature. Even at a safe temperature of around 60 degrees Celsius, the viscosity is still as high as 20 to 100 cSt. Additionally, the inhomogeneity of pyrolysis oil is another problem when removing metals from the pyrolysis oil.

Embodiments of this disclosure address ways to make pyrolysis oil homogenous and less viscous at room temperature, and ways to remove metals from pyrolysis oil at room temperature.

### SUMMARY

Embodiments of the disclosure relate to removing metals from pyrolysis oil. One embodiment is a method for removing metals from pyrolysis oil comprising combining pyrolysis oil and an organic solvent to form a pyrolysis oil mixture and removing metals from the pyrolysis oil mixture to produce a reduced metal content pyrolysis oil mixture. The metals can be removed from the pyrolysis oil mixture comprises using an acidic ion-exchange resin, such as a strongly acid ion-exchange resin. In embodiments, the pyrolysis oil and organic solvent may be mixed together prior to metal removal, and the mixing may be active or passive. The pyrolysis oil mixture can comprise 40-95% pyrolysis oil and 5-60% organic solvent. In embodiments of the disclosure, the organic solvent is ethanol, propanol, ethylene glycol, acetone, and mixtures thereof. In specific embodiments, the pyrolysis oil mixture comprises 5-40%, or 10-20% ethanol, propanol, or combinations thereof. In embodiments, the pyrolysis oil mixture comprises 40-50% pyrolysis oil, 40-50% ethylene glycol, and 5-15% acetone. In certain embodiments, the pyrolysis oil mixture has a viscosity of less than 40 cSt. The pyrolysis oil mixture may be filtered or not filtered prior to metal removal. In some embodiments, the pyrolysis oil mixture does not need to be heated prior to or during metal removal.

The foregoing has outlined rather broadly the features and technical advantages of the present invention in order that the detailed description of the invention that follows may be better understood. Additional features and advantages of the invention will be described hereinafter. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 is a graph showing the viscosity of pyrolysis oil vs. weight percent propanol. The x-axis is the propanol ratio (w/w %) in the mixture, while the y-axis is the viscosity at 40° C. in cSt.

FIG. 2 is a graph showing the equilibrium isotherm results of a pyrolysis oil sample in an acetone-ethylene glycol mixed solvent with an acidic ion-exchange resin. The x-axis is the weight resin over the weight pyrolysis oil in percent, while the y-axis is the percentage of metal removal for five metals. The metals in the graph are as follows: K (dash), Na (circle), Ca (diamond), Mg (triangle), and Fe (square).

FIG. 3 is a graph showing the removal of potassium using three different acidic ion exchange resins. The x-axis shows the weight resin over the weight pyrolysis oil in percent. The y-axis is the percentage of metal removal. The different resins are represented by three different labels; triangle represents Acidic Resin A, diamond represents Acidic Resin B, and square represents Acidic Resin C.

FIG. 4 is a graph showing the removal of sodium using three different acidic ion exchange resins. The x-axis shows the weight resin over the weight pyrolysis oil in percent. The y-axis is the percentage of metal removal. The different resins are represented by three different labels; triangle represents Acidic Resin A, diamond represents Acidic Resin B, and square represents Acidic Resin C.

FIG. 5 is a graph showing the removal of calcium using three different acidic ion exchange resins. The x-axis shows the weight resin over the weight pyrolysis oil in percent. The y-axis is the percentage of metal removal. The different resins are represented by three different labels; triangle represents Acidic Resin A, diamond represents Acidic Resin B, and square represents Acidic Resin C.

FIG. 6 is a graph showing the removal of magnesium using three different acidic ion exchange resins. The x-axis shows the weight resin over the weight pyrolysis oil in percent. The y-axis is the percentage of metal removal. The different resins are represented by three different labels; triangle represents Acidic Resin A, diamond represents Acidic Resin B, and square represents Acidic Resin C.

FIG. 7 is a graph showing the removal of iron using three different acidic ion exchange resins. The x-axis shows the weight resin over the weight pyrolysis oil in percent. The y-axis is the percentage of metal removal. The different resins are represented by three different labels; triangle represents Acidic Resin A, diamond represents Acidic Resin B, and square represents Acidic Resin C.

### DETAILED DESCRIPTION

Embodiments of the disclosure relate to a method to remove metals from pyrolysis oil. Specifically, embodiments of the method relate to using a solvent to reduce the viscosity and increase the homogeneity of pyrolysis oil. Additional embodiments include removal of metals from the pyrolysis oil with acidic ion-exchange.

As used herein, the term “equal” refers to equal values or values within the standard of error of measuring such values. The term “substantially equal” or “about” refers to an amount that is within 5% of the value recited.

As used herein, “a” or “an” means “at least one” or “one or more” unless otherwise indicated. Additionally, “metal removal” refers to removal of one or more type of metal.

Pyrolysis Oil

“Pyrolysis oil,” as used herein, refers to the liquid oil created from the thermal decomposition of carbon based solid material in an oxygen-deprived environment. The carbon based solid material can be, for example, biomass, waste plastics, and coal. Any pyrolysis oil that contains metals can be used in embodiments of this disclosure.

Pyrolysis oil can be created from either a slow or fast pyrolysis processes, with the fast process favoring the production of pyrolysis oil and the slow process favoring the production of biochar. Fast pyrolysis generally relates to the rapid heating of the feedstock material in an oxygen deprived environment to around 300-600° C. with a short residence time of around 0.3-5 seconds. Faster pyrolysis can operate at atmospheric pressures and the production of pyrolysis oil can exceed 60% of the products. Different kinds of reactors can be used in the fast pyrolysis production methods, including but not limited to, bubbling fluidized bed, circulating fluidized beds, ablative pyrolysis, and vacuum pyrolysis. Embodiments of the disclosure include the use of pyrolysis oil which has come from any pyrolysis method.

Embodiments of the disclosure include a mixture comprising pyrolysis oil and a solvent, such as ethanol, acetone, propanol, or ethylene glycol. Such mixtures have lower viscosities and improved homogeneity, making metal removal through the use of acidic-ion exchange resins easier. In certain embodiments, the mixture comprises about 40-95%, about 50-90%, about 60-88%, or about 75-85% by weight percent pyrolysis oil and about 5-50%, about 10-40%, about 12-30%, about 15-25% by weight percent solvent. In a specific embodiment, the mixture comprises about 85% pyrolysis oil. The mixture can also have a viscosity of less than 60, less than 55, less than 50, less than 45, less than 40, less than 35, less than 30, less than 25, less than 20, less than 15, or less than 10 cSt at 20° C. In some embodiments, the mixture has a viscosity of about 5-60, about 10-50, about 15-30 cSt at 20° C.

Solvent System

Embodiments of the disclosure comprise using an organic solvent or solvent mixture is an alternative way to make pyrolysis oil homogeneous and less viscous to allow the further treatments implemented at room temperature. Embodiments of the disclosure include organic solvents which have at least one of the following properties (1) miscible with pyrolysis oil, (2) no inter-reaction with pyrolysis oil, (3) no negative effects on pyrolysis oil further treatments in refineries, (4) low viscosity, (5) low volatility, (6) cost effective, (7) environmentally friendly, (8) safe to handle, and (9) recyclable. As used herein, “organic solvent,” refers to a liquid that dissolves the pyrolysis oil resulting in a lower viscosity solution. In embodiments, the solvent is ethanol, propanol, ethylene glycol, acetone, or mixtures thereof.

In an embodiment, propanol, ethanol, or mixtures thereof are used as a solvent for metal removal processes from pyrolysis oil. The addition of ethanol, for example, improves the homogeneity and reduces the viscosity of the pyrolysis oil sample down to appropriate levels and makes the process operable at and around room temperature. Specific embodiments of the disclosure include mixtures comprising pyrolysis oil and about 5-50%, about 10-40%, about 12-30%, or about 15-25% by weight percent ethanol, propanol, or mixtures thereof. In a specific embodiment, the mixture comprises pyrolysis oil and about 15% by weight percent ethanol, propanol, or mixtures thereof

Ethylene glycol (IUPAC name: ethane-1,2-diol) is an organic compound of the formula HO—CH₂—CH₂—OH. Ethylene glycol is a commercially available solvent which meets almost all of the above listed properties. Acetone (propanone) is an organic compound with the chemical formula (CH₃)₂CO. Embodiments of the disclosure include a mixture comprising pyrolysis oil, acetone, and ethylene glycol. This mixture is found to have a lower viscosity which improves the metal removal process. Embodiments of the disclosure include mixtures comprising between 10-70% pyrolysis oil, 10-50% ethylene glycol, and 5-20% acetone. In specific embodiments of the disclosure, the mixtures comprise 40-50% pyrolysis oil, 40-50% ethylene glycol, and 5-15% acetone.

Metal Removal

Embodiments of the disclosure have been found suitable for further dissolving pyrolysis oil and then removing metals from the pyrolysis oil. As discussed above, the mixtures of pyrolysis oil and solvent are used to improve the homogeneity and to reduce the viscosity of the pyrolysis oil, thus, enabling the efficient removal of metals without the need to heat the pyrolysis oil. For example, the pyrolysis oil solvent mixture can undergo metal removal at less than 100° C., less than 80° C., less than 70° C., less than 60° C., less than 50° C., less than 40° C., less than 30° C. or around 20° C. In certain embodiments, the pyrolysis oil solvent mixture can undergo metal removal between 15° C.-100° C., 15° C.-30° C., 20° C.-60° C., or 20° C.-30° C. In a specific embodiment, the pyrolysis oil solvent mixture can undergo metal removal at room temperature, such as about 23° C.

It is found that the metals in the above described solvent pyrolysis oil mixtures can be removed with acid ion-exchange resins. Adding acetone into pyrolysis oil-ethylene glycol solution makes a less viscous and easier to pass through selected filtration media, such as filter paper, at room temperature under one atmosphere or less pressure to separate adsorbent/resin from the solution. Example 2 evaluates different acidic ion-exchange resins for removing metals from the pyrolysis oil and ethanol mixtures.

Embodiments of the disclosure include removing metals from a mixture comprising pyrolysis oil and a solvent. Metal removal can be accomplished through the use of acidic ion-exchange resins. In specific embodiments, the acid ion exchange resins are strongly acidic ion-exchange resins. The manufactures of ion exchange resins mark the resins as strong or weak. The resins may be used free in the mixture, or may be implemented in a column. Additionally, the metal removal process may be implemented at temperatures between the ranges of 15° C.-40° C., around room temperature, or around 21° C.

### EXAMPLES

The following examples are included to demonstrate specific embodiments of the disclosure. It should be appreciated by those of skill in the art that the techniques disclosed in the examples that follow represent techniques discovered by the inventors to function well in the practice of the invention, and thus, can be considered to constitute modes for its practice. However, those skilled in the art should, in light of the present disclosure, appreciate that many changes can be made in the specific embodiments disclosed and still obtain a like or similar result without departing from the spirit and scope of the invention.

### Example 1

The viscosities of pure pyrolysis oil and mixtures of pyrolysis oil+ethylene glycol, and pyrolysis oil+ethylene glycol+acetone were compared. Table 1 shows that with adding 10% acetone, the viscosity is reduced by about 50% more from pyrolysis oil+ethylene glycol.

TABLE 1
Viscosities of a sample pyrolysis oil-solvent mixture, cSt

| | Example Pyrolysis Oil | | |
|---|---|---|---|
| Temperature | | 50% Oil + | 45% Oil + |
| ° C. | 100% Oil | 50% Glycol | 45% Glycol + 10% Acetone |
| 20 | 425 | 46.8 | 22.8 |
| 40 | 51.0 | 17.2 | 9.4 |
| 60 | 19.7 | 7.7 | 4.8 |

45% pyrolysis oil:45% ethylene glycol:10% acetone was found to be a good mixture for reducing the viscosity in the pyrolysis oil used in this example. The viscosity at 20 degrees Celsius for such a mixture is almost the same as the “pure” pyrolysis oil viscosity at 60 degrees Celsius. The measured viscosities of pyrolysis oil, pyrolysis oil/ethylene glycol solution, and pyrolysis oil/ethylene glycol/acetone solution at three different temperatures are listed above in Table 1. Such a solution makes not only the pyrolysis oil homogeneous and stable, but also makes the metal removal process operable at room temperature with no heating needed.

FIG. 2 shows the equilibrium isotherm study result with an acidic ion-exchange resin. The study was conducted with pyrolysis oil samples in ethylene glycol and acetone solution at room temperature. Five metal contents, calcium, iron, potassium, magnesium, and sodium, were measured. Sodium can be removed with 0.5% (in weight) acidic ion-exchange resin. Three of the five metals were removed at the weight ratio of 1.2%, resin to oil W_{f}/W_{O}, while iron used about 3.3%. In this example, the resin was put free into the mixture and shaken for less than 6 hours, but could have also been run in a column. The mixture then underwent vacuum filtration to separate the resin from the oil. 20 grams of pyrolysis oil and solvent mixture was used in each mixtures.

### Example 2

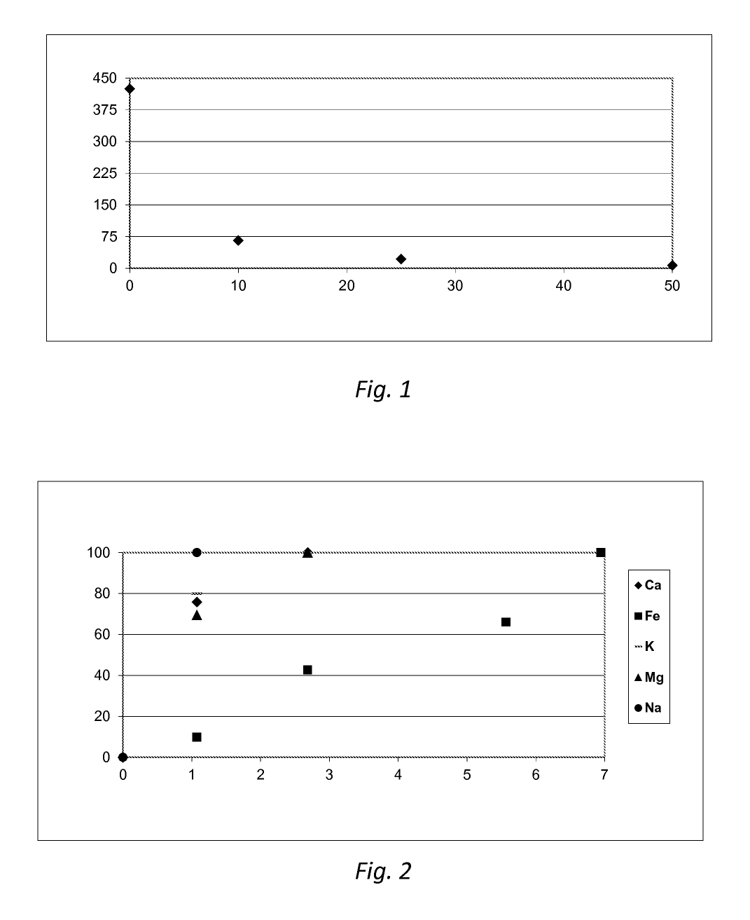

Propanol was also chosen as a solvent for this example to demonstrate the metal removal process from pyrolysis oil. A 15 weight % addition of propanol was found to reduce the viscosity of the pyrolysis oil sample down to an appropriate level (FIG. 1) and make the process operable at room temperature.

A term of “percentage of removal” was used to evaluate the efficiency of metal removal. It is calculated by Equation 1, below. Calculated percentage of removal value would be 100% when the concentration measured by ICP is 5 ppm or less.

Percentage of removal=100×(Cₒ−C)/Cₒ (%) Equation-1

Where Cₒ is the initial concentration, in ppm, of the element in the raw pyrolysis oil sample, C is the concentration, in ppm, of the element in the treated pyrolysis oil sample

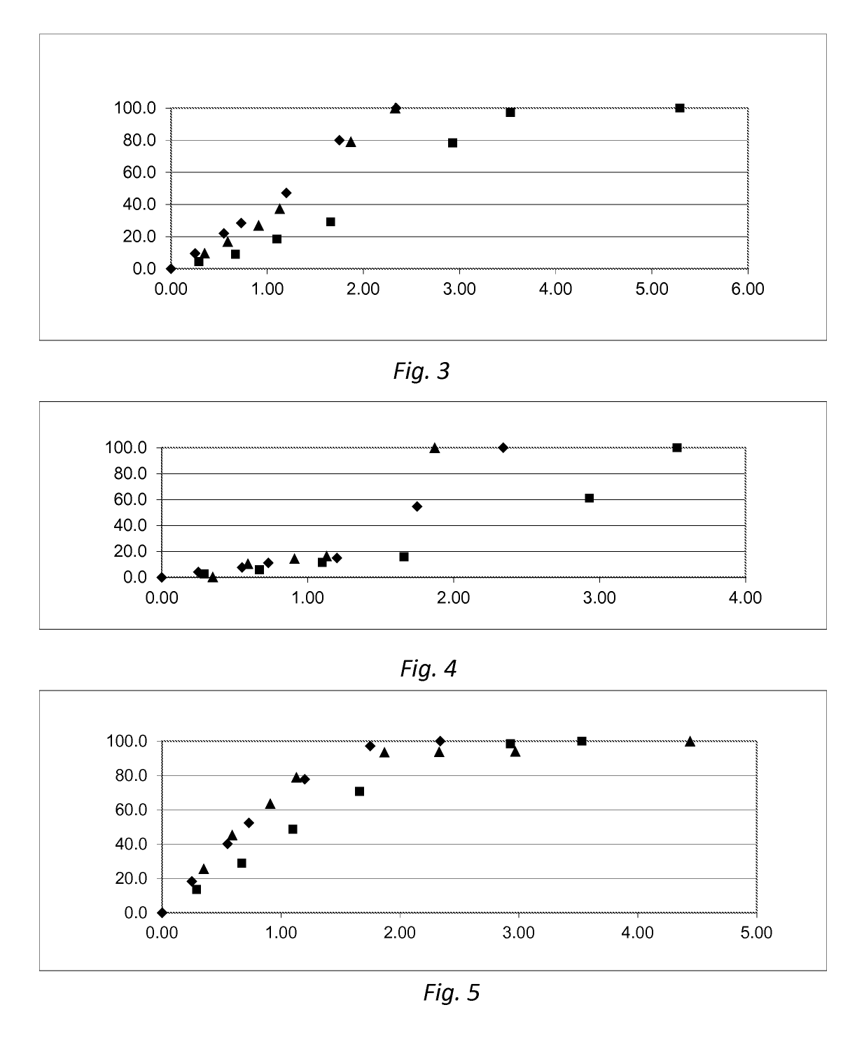

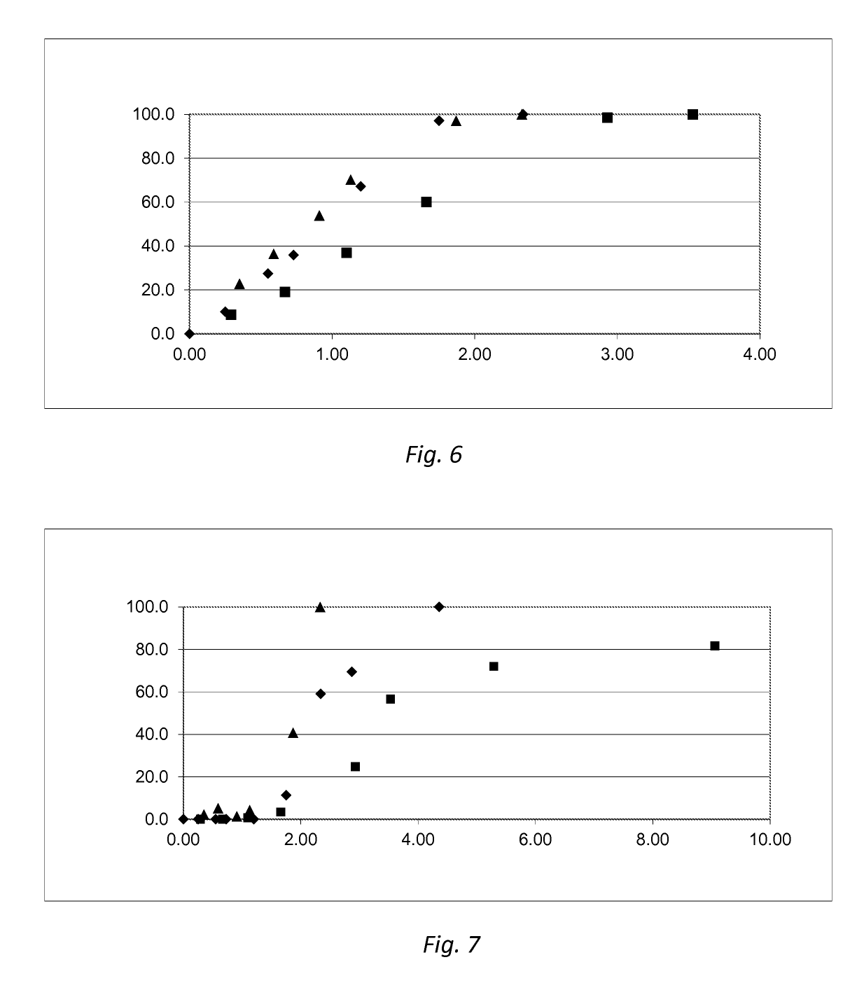

In order to determine which type of adsorbents or resins is an appropriate material for removing potassium (K), sodium (Na), calcium (Ca), magnesium (Mg), and iron (Fe) from pyrolysis oil, fifteen (15) adsorbents and resins were investigated with an equilibrium isotherm study. The results of three acidic ion-exchange resin runs for each metal are shown in FIGS. 3-7.

### Example 2

The viscosities of a sample pyrolysis oil blended with 1) ethanol, 2) propanol and 3) a 1:1 mixture of ethanol and propanol was measured at 20° C. as shown below in Table 2.

TABLE 2
Viscosities (cSt) for an example pyrolysis oil and varies solvents

| Mix Ratio, w/w % | | | | |
|---|---|---|---|---|
| | | 1:1 Ethanol and | | Viscosity |
| | | Propanol | | at 20° C., |
| Ethanol | Propanol | Mixture | Pyrolysis Oil | cSt |
| 0 | | | 100 | 424.6 |
| 10 | | | 90 | 65.94 |
| 25 | | | 75 | 21.66 |
| 50 | | | 50 | 7.087 |
| | 10 | | 90 | 78.43 |
| | 25 | | 75 | 32.87 |
| | 50 | | 50 | 12.20 |
| 5 | 5 | 10 | 90 | 71.98 |
| 12.5 | 12.5 | 25 | 75 | 26.85 |
| 25 | 25 | 50 | 50 | 9.231 |

### REFERENCES

### US2012/0317871

Finish Thompson Inc. Recovery of waste engine coolants using advanced vacuum distillation technology.

What is claimed is:

1. A method for removing metals from pyrolysis oil comprising
combining pyrolysis oil and an organic solvent to form a pyrolysis oil mixture; and
removing metal from the pyrolysis oil mixture to produce a reduced metal content pyrolysis oil mixture.

2. The method of claim 1, wherein removing the metal from the pyrolysis oil mixture comprises using an acidic ion-exchange resin.

3. The method of claim 2, wherein the acidic ion-exchange resin is a strongly acid ion-exchange resin.

4. The method of claim 1, further comprising mixing the pyrolysis oil and the organic solvent together prior to metal removal.

5. The method of claim 4, wherein the mixing is active or passive.

6. The method of claim 1, wherein the pyrolysis oil mixture comprises 40-95% pyrolysis oil and 5-60% organic solvent.

7. The method of claim 1, wherein the organic solvent is ethanol, propanol, or mixtures thereof

8. The method of claim 7, wherein the pyrolysis mixture comprises 5-40% ethanol.

9. The method of claim 8, wherein the pyrolysis mixture comprises 10-20% ethanol.

10. The method of claim 1, wherein the organic solvent comprises ethylene glycol and acetone.

11. The method of claim 10, wherein the pyrolysis oil mixture comprises 40-50% pyrolysis oil, 40-50% ethylene glycol, and 5-15% acetone.

12. The method of claim 1, wherein the pyrolysis oil mixture has a viscosity of less than 40 cSt.

13. The method of claim 1, wherein the pyrolysis mixture is filtered prior to metal removal.

14. The method of claim 1, wherein the pyrolysis mixture is not filtered prior to metal removal.

15. The method of claim 1, wherein the pyrolysis oil mixture is not heated prior to or during metal removal.

16. The method of claim 1, wherein the pyrolysis oil mixture is less than 100° C. prior to or during metal removal.
